(19) United States
(12) Patent Application Publication
(10) Pub. No.: US 2024/0261268 A1
(43) Pub. Date: Aug. 8, 2024

(54) METHODS OF SLOWING AN INCREASE IN BRAIN VENTRICULAR VOLUME

(71) Applicant: VANDA PHARMACEUTICALS INC., Washington, DC (US)

(72) Inventors: Maria AIT-TIHYATY, Quebec (CA); Zaid Serhal SAAD, San Diego, CA (US); Ritobrato DATTA, San Diego, CA (US); Allitia DIBERNARDO, Titusville, NJ (US); Hartmuth KOLB, San Diego, CA (US); Frank WIEGAND, San Francisco, CA (US)

(21) Appl. No.: 18/565,752

(22) PCT Filed: Feb. 10, 2023
(86) PCT No.: PCT/EP2023/053304

§ 371 Date: Nov. 30, 2023

Related U.S. Application Data
(60) Provisional application No. 63/309,250, filed on Feb. 11, 2022.

(51) Int. Cl. A61K 31/426; A61P 25/00

(52) U.S. Cl. CPC A61K 31/426; A61P 25/00

(57) ABSTRACT

The disclosure relates to methods of slowing an increase in brain ventricular volume. In certain aspects, methods of slowing an increase in brain ventricular volume in a patient with multiple sclerosis are disclosed.

### TECHNICAL FIELD

The present disclosure relates to methods of slowing an increase in brain ventricular volume (hereinafter also referred to as ventricular volume or VV), and, in particular, methods of slowing an increase in brain ventricular volume in a patient having multiple sclerosis (MS).

### BACKGROUND

Multiple sclerosis (MS) is a chronic autoimmune inflammatory disease of the central nervous system affecting approximately 2.5 million people worldwide. The disease is clinically perceived by relapses and progressive loss of neurological function, primarily attributed to demyelination, axonal loss, and gliosis culminating in long-term multifocal sclerotic plaques in the brain and spinal cord leading to neurological impairment and severe disability. The two main subtypes of MS are relapsing forms of MS (RMS) which represent 85% of MS patients and include relapsing-remitting disease (RRMS), clinically isolated syndrome, and active secondary progressive disease; and primary progressive MS (PPMS) which affects only 15% of MS patients.

Previous radiological and pathological studies have established that whole brain and spinal cord atrophy occur in multiple sclerosis (MS) in excess of that expected with age. While the mechanisms of atrophy in MS are not well understood, it probably indicates loss of functionally important structures: myelin and axons that are the majority bulk of brain white matter, although variable effects on tissue volumes may also arise from glial cell proliferation or loss, gliosis, inflammation, and oedema.

Brain ventricle enlargement is commonly linked to neurodegeneration and brain atrophy. While this occurs during the course of normal aging, shrinkage of the cortex and expansion of the ventricles proceeds more rapidly in MS patients.

Hence, there persists a need for new products and methods that can reduce the rate of ventricular volume increase while being safe and well tolerated.

### SUMMARY

In embodiments, the present disclosure is directed to a method of slowing an increase in brain ventricular volume in a patient in need thereof, comprising administering to the patient ponesimod using a regimen that is effective to slow an increase in brain ventricular volume.

In embodiments, the present disclosure is directed to a method of slowing an increase in brain ventricular volume in a patient in need thereof, comprising assessing cognitive deficiencies or physical deficiencies in the patient; and administering ponesimod to the patient using a regimen that is effective to slow an increase in ventricular volume.

In embodiments, the present disclosure is directed to a method of slowing an increase in brain ventricular volume in a patient in need thereof, comprising assessing brain ventricular volume prior to ponesimod administration; and administering ponesimod to the patient using a regimen that is effective to slow an increase in brain ventricular volume.

In embodiments, the present disclosure is directed to a method of slowing an increase in brain ventricular volume in a patient in need thereof, comprising administering ponesimod to the patient using a regimen that is effective to slow an increase in brain ventricular volume relative to a patient having substantially similar baseline disease characteristics and receiving a standard of care treatment that does not comprise ponesimod.

In some aspects, the present disclosure is directed to ponesimod for use in a method of slowing an increase in brain ventricular volume in a patient in need thereof, said method comprising administering ponesimod to the patient using a regimen that is effective to slow an increase in brain ventricular volume.

In some aspects, the present disclosure is directed to the use of ponesimod in the preparation of a medicament for slowing an increase in brain ventricular volume in a patient in need thereof, wherein said medicament is adapted to be administered to the patient using a regimen that is effective to slow an increase in brain ventricular volume.

In certain aspects, the methods of the disclosure are performed on a human patient suffering from multiple sclerosis. In some embodiments, the patient's multiple sclerosis is relapsing multiple sclerosis. In other embodiments, the relapsing multiple sclerosis comprises relapsing-remitting disease, clinically isolated syndrome or active secondary progressive disease.

### BRIEF DESCRIPTION OF THE DRAWINGS

FIG. 1 shows the design of the study described in Example 1.

FIG. 2 shows the 12-lead electrocardiogram (ECG) heart rate and absolute change from pre-dose at Day 1, by hour (Analysis Set: Safety Set).

FIG. 3 shows ventricular volume change for the 3rd ventricle and the lateral ventricle at week 60 and week 108 compared to baseline for treatment with ponesimod and with teriflunomide.

FIG. 4 shows the longitudinal increase in the size of the 3rd ventricle at week 60 and week 108 compared to baseline for treatment with ponesimod and with teriflunomide.

FIG. 5 shows the longitudinal increase in the size of the left lateral ventricle at week 60 and week 108 compared to baseline for treatment with ponesimod and with teriflunomide.

FIG. 6 shows the longitudinal increase in the size of the right lateral ventricle at week 60 and week 108 compared to baseline for treatment with ponesimod and with teriflunomide.

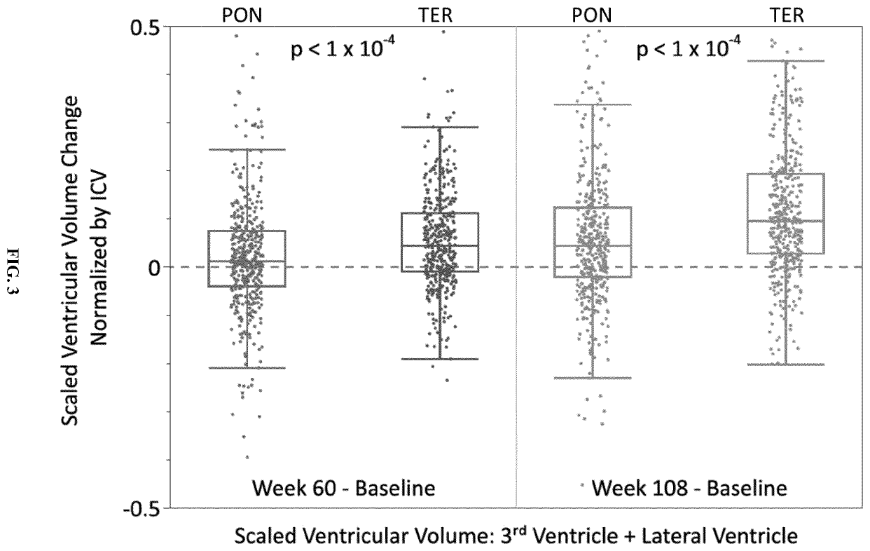

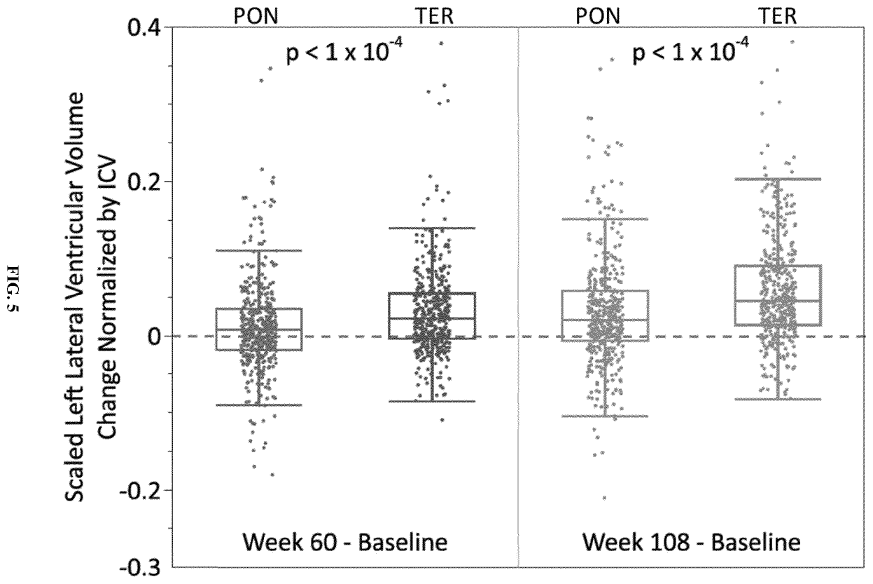

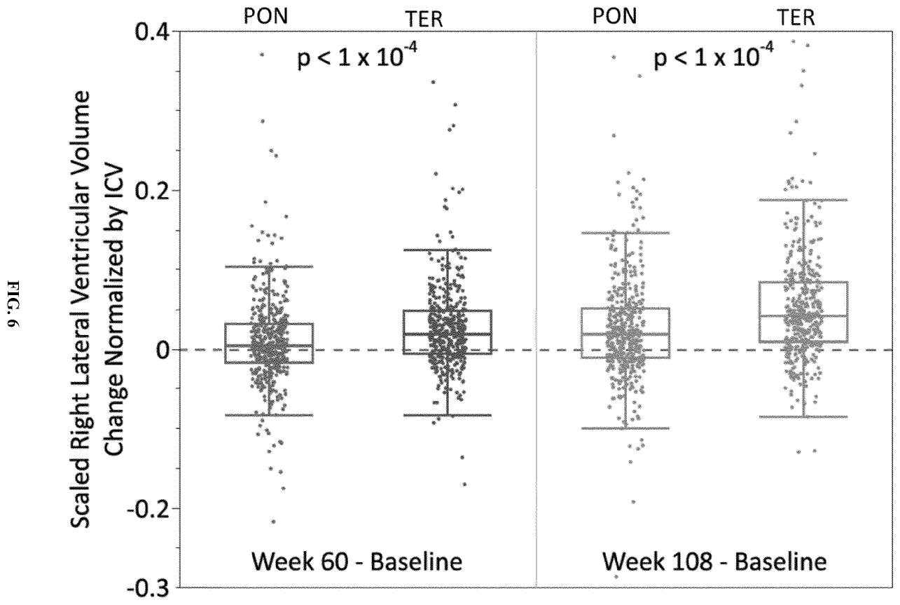

In FIGS. 3-6, ICV stands for intracranial volume and the p-values are uncorrected from a t-test comparing the treatment groups.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosure which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

In some aspects, the present disclosure is directed to a method of slowing an increase in ventricular volume in a patient in need thereof, comprising administering to the patient ponesimod using a regimen that is effective to slow an increase in ventricular volume.

In some aspects, the present disclosure is directed to a method of slowing an increase in ventricular volume in a patient in need thereof, comprising assessing cognitive deficiencies or physical deficiencies in the patient; and administering ponesimod to the patient using a regimen that is effective to slow an increase in ventricular volume.

In embodiments, the present disclosure is directed to a method of slowing an increase in brain ventricular volume in a patient in need thereof, comprising assessing brain ventricular volume prior to ponesimod administration; and administering ponesimod to the patient using a regimen that is effective to slow an increase in brain ventricular volume.

In some aspects, the assessing brain ventricular volume prior to ponesimod administration comprises assessment by MRI (Magnetic Resonance Imaging). In embodiments, the assessment by MRI prior to ponesimod administration comprises baseline MRI assessment. In embodiments, the assessment by MRI prior to ponesimod administration comprises longitudinal MRI assessment. MRI assessment may also take place during or after ponesimod administration in order to assess results and outcomes.

In some aspects, the present disclosure is directed to a method of slowing an increase in ventricular volume in a patient in need thereof, comprising administering ponesimod to the patient using a regimen that is effective to slow an increase in ventricular volume relative to a patient having substantially similar baseline characteristics and receiving a standard of care treatment that does not comprise ponesimod.

In some aspects, the ventricular volume is determined by magnetic resonance imaging. In some aspects, the ventricular volume determined by magnetic resonance imaging is determined at baseline. In some aspects, the ventricular volume determined by magnetic resonance imaging is determined after a period of treatment with a drug.

In some aspects, the methods of the disclosure are performed on a human patient suffering from multiple sclerosis. In some embodiments, the patient's multiple sclerosis is relapsing multiple sclerosis. In other embodiments, the relapsing multiple sclerosis comprises relapsing-remitting disease, clinically isolated syndrome or active secondary progressive disease.

In some aspects, the present disclosure is directed to ponesimod in combination with an additional therapeutic agent. For example, the therapeutic agent may be an agent that reduces or normalizes the increase in ventricular volume in the patient. In some aspects, the additional therapeutic agent is teriflunomide, leflunomide, methyl fumarate, dimethyl fumarate, (N,N-diethylcarbamoyl)methyl methyl (2E)but-2-ene-1,4-dioate, or 2-(2,5-dioxopyrrolidin-1-yl)ethyl methyl (2E)but-2-ene-1,4-dioate.

In some aspects, cognitive deficiencies are assessed and include, for example, information processing; memory; attention/concentration; executive functions; visuospatial functions; or verbal fluency. In certain aspects, the information processing deficiencies comprise deficiencies associated with information gathered by the five senses. In certain aspects, the memory deficiencies comprise deficiencies associated with acquiring, retaining and retrieving new information. In certain aspects, the executive functions deficiencies comprise deficiencies associated with planning and prioritizing. In certain aspects, the visuospatial functions deficiencies comprise deficiencies associated with visual perception and constructional abilities. In certain aspects, the verbal fluency deficiencies comprise deficiencies associated with word-finding. Examples of tests for assessing cognitive deficiencies are described in Example 1.

In some aspects, the physical deficiencies are vision, hearing, speaking, swallowing, breathing, muscle weakness, hand-eye coordination, balance and gait. In certain aspects, the vision deficiencies comprise double vision, blurriness, pain, and problems seeing contrast. In certain aspects, the hearing deficiencies comprise hearing loss and deafness. In certain aspects, the speaking deficiencies comprise slurring, poor articulation and volume control issues. In certain aspects, the muscle weakness comprises pain, tingling, and numbness of the arms and legs. Examples of tests for assessing physical deficiencies are described in Example 1.

Given the slowing of an increase in ventricular volume resulting from the methods disclosed herein, a treating physician has additional treatment options. For example, if an assessment indicates a high degree of cognitive or physical deficiencies, a patient can be administered ponesimod as opposed to other standard of care options. In addition, if a patient currently receiving a standard of care treatment is experiencing a high degree of cognitive or physical deficiencies, the treating physician may transition the patient to a ponesimod treatment regimen.

In some aspects, the patient has a baseline expanded disability status scale (EDSS) score of less than or equal to 3.5. In some aspects, the patient has a baseline expanded disability status scale (EDSS) score of less than or equal to 3.0.

In some aspects, the regimen is effective to slow an increase in ventricular volume by at least about 20% to about 80% relative to a patient having substantially similar baseline disease characteristics and receiving a standard of care treatment that does not comprise ponesimod. Such a relative analysis is disclosed in Example 1.

In some aspects, the regimen is effective to slow an increase in ventricular volume by at least about 25% to about 75% relative to a patient having substantially similar baseline disease characteristics and receiving a standard of care treatment that does not comprise ponesimod.

In some aspects, the regimen is effective to slow an increase in ventricular volume by at least about 30% to about 70% relative to a patient having substantially similar baseline disease characteristics and receiving a standard of care treatment that does not comprise ponesimod.

In some aspects, the regimen is effective to slow an increase in ventricular volume by at least about 35% to about 65% relative to a patient having substantially similar baseline disease characteristics and receiving a standard of care treatment that does not comprise ponesimod.

In some aspects, the regimen is effective to slow an increase in ventricular volume by at least about 40% to about 60% relative to a patient having substantially similar baseline disease characteristics and receiving a standard of care treatment that does not comprise ponesimod.

In some aspects, the regimen is effective to slow an increase in ventricular volume by at least about 45% to about 55% relative to a patient having substantially similar baseline disease characteristics and receiving a standard of care treatment that does not comprise ponesimod.

In some aspects, the regimen is effective to slow an increase in ventricular volume by at least about 20% relative to a patient having substantially similar baseline disease characteristics and receiving a standard of care treatment that does not comprise ponesimod. In some aspects, the relative slowing of an increase in ventricular volume is at least about 25%; or at least about 30%; or at least about 35%; or at least about 40%; or at least about 45%; or at least about 50%; or at least about 55%; or at least about 60%; or at least about 65%; or at least about 70%; or at least about 75%; or at least about 80% relative to a patient having substantially similar baseline disease characteristics and receiving a standard of care treatment that does not comprise ponesimod.

In some aspects, the regimen is effective to slow an increase in ventricular volume by about 20% to about 80% relative to a patient having substantially similar baseline disease characteristics and receiving a standard of care treatment that does not comprise ponesimod. In some aspects, the relative slowing of an increase in ventricular volume is about 20% to about 25%; or about 25% to about 30%; or about 30% to about 35%; or about 35% to about 40%; or about 40% to about 45%; or about 45% to about 50%; or about 50% to about 55%; or about 55% to about 60%; or about 60% to about 65%; or about 65% to about 70%; or about 70% to about 75%; or about 75% to about 80% relative to a patient having substantially similar baseline disease characteristics and receiving a standard of care treatment that does not comprise ponesimod.

Typically, the relative slowing of an increase in ventricular volume demonstrated by the methods disclosed herein results after at least about 30-90 weeks from initiation of treatment with ponesimod; or after at least about 35-85 weeks from initiation of treatment with ponesimod; or after at least about 40-80 weeks from initiation of treatment with ponesimod; or after at least about 45-75 weeks from initiation of treatment with ponesimod; or after at least about 50-70 weeks from initiation of treatment with ponesimod; or after at least about 55-65 weeks from initiation of treatment with ponesimod; or after at least about 60 weeks from initiation of treatment with ponesimod.

In some aspects, the relative slowing of an increase in ventricular volume demonstrated by the methods disclosed herein results after at least about 30 weeks from initiation of treatment with ponesimod; or after at least about 35 weeks; or after at least about 40 weeks; or after at least about 45 weeks; or after at least about 50 weeks; or after at least about 55 weeks; or after at least about 60 weeks; or after at least about 65 weeks; or after at least about 70 weeks; or after at least about 75 weeks; or after at least about 80 weeks; or after at least about 85 weeks; or after at least about 90 weeks from initiation of treatment with ponesimod.

In some aspects, the relative slowing of an increase in ventricular volume demonstrated by the methods disclosed herein results after at least about 30-35 weeks from initiation of treatment with ponesimod; or after at least about 35-40 weeks; or after at least about 40-45 weeks; or after at least about 45-50 weeks; or after at least about 50-55 weeks; or after at least about 55-60 weeks; or after at least about 60-65 weeks; or after at least about 65-70 weeks; or after at least about 70-75 weeks; or after at least about 75-80 weeks; or after at least about 80-85 weeks; or after at least about 85-90 weeks; or after at least about 90 weeks from initiation of treatment with ponesimod.

Typically, the relative slowing of an increase in ventricular volume demonstrated by the methods disclosed herein results after at least about 90-120 weeks from initiation of treatment with ponesimod; or after at least about 95-115 weeks from initiation of treatment with ponesimod; or after at least about 100-110 weeks from initiation of treatment with ponesimod; or after at least about 105-110 weeks from initiation of treatment with ponesimod; or after at least about 108 weeks from initiation of treatment with ponesimod.

In some aspects, the relative slowing of an increase in ventricular volume demonstrated by the methods disclosed herein results after at least about 90 weeks from initiation of treatment with ponesimod; or after at least about 95 weeks; or after at least about 100 weeks; or after at least about 105 weeks; or after at least about 110 weeks; or after at least about 115 weeks; or after at least about 120 weeks from initiation of treatment with ponesimod.

In some aspects, the relative slowing of an increase in ventricular volume demonstrated by the methods disclosed herein results after at least about 90-95 weeks from initiation of treatment with ponesimod; or after at least about 95-100 weeks; or after at least about 100-105 weeks; or after at least about 105-110 weeks; or after at least about 110-115 weeks; or after at least about 115-120 weeks; or after at least about 108 weeks from initiation of treatment with ponesimod.

In certain aspects, the patient has a neurodegenerative disease other than multiple sclerosis. In certain aspect the patient has Alzheimer's disease. In certain aspects, the patient has Parkinson's disease.

As used herein, the term “ponesimod” refers to the compound (R)-5-[3-chloro-4-(2,3-dihydroxy-propoxy)-benz[Z]ylidene]-2-([Z]-propylimino)-3-o-tolyl-thiazolidin-4-one, which has the following structure:

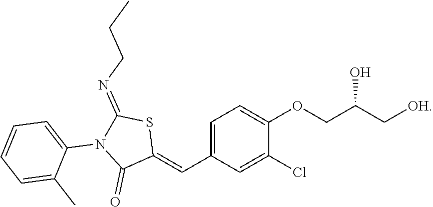 [SMILES: CCC/N=C1\S/C(=C\c2ccc(OC[C@H](O)CO)c(Cl)c2)C(=O)N1c1ccccc1C]

In some embodiments, “ponesimod” also refers to pharmaceutically acceptable salts of ponesimod. The term “pharmaceutically acceptable salt” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example Handbook of Pharmaceutical Salts. Properties, Selection and Use, P. Heinrich Stahl, Camille G. Wermuth (Eds.), Wiley-VCH, 2008 and Pharmaceutical Salts and Co-crystals, Johan Wouters and Luc Quéré (Eds.), RSC Publishing, 2012.

It is to be understood that the present disclosure encompasses ponesimod in any form including amorphous as well as crystalline forms. It is further to be understood that crystalline forms of ponesimod encompasses all types of crystalline forms including polymorphs, solvates and hydrates, salts and co-crystals (when the same molecule can be co-crystallized with different co-crystal formers) provided they are suitable for pharmaceutical administration. In some embodiments, ponesimod is in crystalline form A or crystalline form C as described in WO 2010/046835, incorporated herein by reference. In some embodiments, ponesimod is in crystalline form C.

It should be noted that the amounts of ponesimod described herein are set forth on a ponesimod free base basis. That is, the amounts indicate that amount of the ponesimod molecule administered, exclusive of, for example, solvent (such as in solvates) or counterions (such as in pharmaceutically acceptable salts).

In some embodiments, the effective regimen comprises a daily dose of ponesimod. In some embodiments, the daily dose of ponesimod is administered orally.

In some embodiments, the daily dose of ponesimod is administered once daily.

In some embodiments, the daily dose of ponesimod is about 15 to about 25 mg. In further embodiments, the daily dose of ponesimod is about 15 mg, about 16 mg, about 17 mg, about 18 mg, about 19 mg, about 20 mg, about 21 mg, about 22 mg, about 23 mg, about 24 mg, or about 25 mg. In certain embodiments, the daily dose of ponesimod is about 20 mg.

In some embodiments, about 20 mg of ponesimod is administered orally once daily.

In other embodiments, the effective regimen comprises an up-titration, followed by a daily maintenance dose of ponesimod. An up-titration is a dosing procedure in which the daily dose of ponesimod is gradually increased over a period of days, culminating with administration of the maintenance dose.

In some embodiments, the regimen comprises an up-titration at the initiation of the method of the disclosure. In other embodiments, the regimen comprises an up-titration upon re-initiation of the method after a discontinuation of the method of the disclosure. As used herein, “upon re-initiation of the method after a discontinuation” means an interruption of the administration of ponesimod of at least one, at least two or preferably at least 3 days before treatment is re-initiated. In some embodiments, the regimen comprises an up-titration step at initiation of the method or upon re-initiation of the method after a discontinuation.

In some embodiments of the methods of the disclosure, the up-titration regimen one disclosed in U.S. Pat. No. 10,220,023, incorporated herein by reference. For example, in certain aspects, the up-titration comprises administering orally once daily about 2 mg of ponesimod on days 1 and 2; about 3 mg of ponesimod on days 3 and 4; about 4 mg of ponesimod on days 5 and 6; about 5 mg of ponesimod on day 7; about 6 mg of ponesimod on day 8; about 7 mg of ponesimod on day 9; about 8 mg of ponesimod on day 10; about 9 mg of ponesimod on day 11; and about 10 mg of ponesimod on days 12, 13, and 14.

In other embodiments of the methods of the disclosure, the up-titration comprises administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; 9 mg of ponesimod on day 11; and 10 mg of ponesimod on days 12, 13, and 14.

In some embodiments, the maintenance dose is about 20 mg of ponesimod once daily.

In some embodiments, the regimen comprises an up-titration step at initiation of the method or upon re-initiation of the method after a discontinuation, comprising administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; and 9 mg of ponesimod on day 11; 10 mg of ponesimod on days 12, 13, and 14, followed by the administering of the 20 mg of ponesimod once daily thereafter.

As used herein, the term “teriflunomide” refers to the compound Z)-2-cyano-3-hydroxy-but-2-enoic acid-(4′-trifluoromethylphenyl)-amide, which has the following structure:

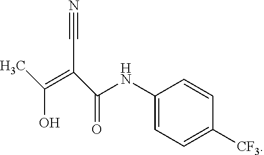 [SMILES: C/C(O)=C(\C#N)C(=O)Nc1ccc(C(F)(F)F)cc1]

In some embodiments, “teriflunomide” also refers to pharmaceutically acceptable salts of teriflunomide. The term “pharmaceutically acceptable salt” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example Handbook of Pharmaceutical Salts. Properties, Selection and Use, P. Heinrich Stahl, Camille G. Wermuth (Eds.), Wiley-VCH, 2008 and Pharmaceutical Salts and Co-crystals, Johan Wouters and Luc Quéré (Eds.), RSC Publishing, 2012.

It is to be understood that the present disclosure encompasses teriflunomide in any form including amorphous as well as crystalline forms. It is further to be understood that crystalline forms of teriflunomide encompasses all types of crystalline forms including polymorphs, solvates and hydrates, salts and co-crystals (when the same molecule can be co-crystallized with different co-crystal formers) provided they are suitable for pharmaceutical administration.

It should be noted that the amounts of teriflunomide described herein are set forth on a teriflunomide free base basis. That is, the amounts indicate that amount of the teriflunomide molecule administered, exclusive of, for example, solvent (such as in solvates) or counterions (such as in pharmaceutically acceptable salts).

Leflunomide (e.g., 5-methyl-N-(4-(trifluoromethyl)phenyl)isoxazole-4-carboxamide) can be used for the treatment of multiple sclerosis. In vivo leflunomide is metabolized to the active metabolite teriflunomide which is responsible for leflunomide's activity in vivo. Leflunomide can be prepared according to procedures known in the art, for example as described in U.S. Pat. No. 4,284,786.

Dimethyl fumarate (e.g., DMF) has been described in WO 00/030622 to be useful for the treatment of autoimmune diseases and Tecfidera® has been approved for the treatment of relapsing forms of multiple sclerosis. Dimethyl fumarate can be prepared according to procedures known in the art for example as described in EP 0312697 A2.

Methyl fumarate (e.g., monomethyl fumarate or MMF) has been shown to be a pharmacologically active metabolite of dimethyl fumarate. Methyl fumarate can be prepared according to procedures known in the art for example as described in EP 0312697 A2.

(N,N-Diethylcarbamoyl)methyl methyl (2E)but-2-ene-1,4-dioate (e.g. XP23829) is a prodrug that is rapidly converted to monomethyl fumarate. XP23829 is currently in clinical development for the treatment of relapsing forms of multiple sclerosis. (N,N-diethyl-carbamoyl)methyl methyl (2E)but-2-ene-1,4-dioate and the preparation thereof is described in WO 2010/022177.

2-(2,5-Dioxopyrrolidin-1-yl)ethyl methyl (2E)but-2-ene-1,4-dioate (e.g., ALKS 8700) is a prodrug that rapidly converts to monomethyl fumarate. ALKS 8700 is currently in clinical development for the treatment of multiple sclerosis. 2-(2,5-Dioxopyrrolidin-1-yl)ethyl methyl (2E)but-2-ene-1,4-dioate and the preparation thereof is described in WO 2014/152494.

As used herein, the term “standard of care treatment” refers to a physician-prescribed treatment, and, in particular a prescribed treatment for MS. In some embodiments, the standard of care comprises, consists of, or consists essentially of administering an MS treatment that has been approved by a regulatory authority. In some embodiments, the standard of care treatment is Interferon (IFN) β-1a 30 mcg i.m. once weekly (Avonex®), IFN β-1a 22 or 44 mcg s.c. 3 times weekly (Rebif®), IFN β-1b 250 mcg s.c. every other day (Betaferon®, Extavia®), Pegylated IFN β-1a 125 mcg subcutaneously every 2 weeks (Plegridy®), Glatiramer acetate 20 mg s.c. once a day (o.d.) or 40 mg subcutaneously 3 times weekly (Copaxone®), Glatiramer acetate 20 mg s.c. o.d. (Glatopa®), Natalizumab 300 mg i.v. every 4 weeks (Tysabri®), Mitoxantrone i.v. every 3 months (Novantrone®), Alemtuzumab concentrate for solution for infusion, 12 mg alemtuzumab in 1.2 mL (10 mg/mL) (Lemtrada®), Fingolimod 0.5 mg orally o.d. (Gilenya®), Teriflunomide 7 mg, 14 mg o.d. (Aubagio®), Dimethyl fumarate (BG-12) gastro-resistant hard capsules 120/240 mg twice daily (Tecfidera®), or Cladribine 40 to 100 mg orally per treatment week (Mavenclad®).

In some embodiments, the standard of care treatment comprises a SIP receptor modulator that is not ponesimod.

In other embodiments, the standard of care treatment comprises teriflunomide. In some embodiments, the standard of care treatment comprises administration of about 14 mg of teriflunomide orally once daily.

With respect to baseline assessments, including baseline disease characteristics, baseline refers to a time period prior to initiation of treatment with ponesimod and/or standard of care treatment. This time period is typically up to about 45 days prior to initiation of treatment with ponesimod and/or standard of care treatment, including, for example, up to about 40 days, up to about 35 days, up to about 30 days, up to about 25 days, up to about 20 days, up to about 15 days, or up to about 10 days prior to initiation of treatment with ponesimod. Examples of baseline disease characteristics are disclosed in Example 1. In other circumstances, assessments can be made during or after treatment with ponesimod and/or standard of care treatment in order to further compare or assess results and outcomes.

In some aspects, the present disclosure is directed to a method of slowing an increase in ventricular volume in a patient in need thereof, wherein the patient is being treated with a first treatment, comprising terminating the first treatment, and administering ponesimod to the patient using a regimen that is effective to slow the increase in ventricular volume.

In some embodiments, the first treatment comprises administration of teriflunomide. In some embodiments, the first treatment comprises administration of about 14 mg of teriflunomide orally once daily.

In some embodiments, the first treatment is terminated and the ponesimod is administered within about 108 weeks from initiation of the first treatment. In some embodiments, the first treatment is terminated and the ponesimod is administered within about 60 weeks from the initiation of the first treatment.

The following Example is provided to illustrate some of the concepts described within this disclosure. While the Example is considered to provide an embodiment, it should not be considered to limit the more general embodiments described herein.

### Example 1

### Study Design

A prospective, multicenter, randomized, double-blind, active controlled, parallel-group, phase III, superiority study was conducted. The study was designed to compare the efficacy, safety, and tolerability of ponesimod 20 mg vs teriflunomide 14 mg in adult subjects with relapsing MS.

Randomization: Subjects were randomized in a 1:1 ratio to ponesimod 20 mg or teriflunomide 14 mg, stratified by prior use of MS disease modifying treatment (DMT) in the last two years prior to randomization (yes, no) and by baseline expanded disability status scale (EDSS) score (EDSS≤3.5, EDSS >3.5).

### Inclusion Criteria

This study enrolled adult male and female subjects aged 18 to 55 years with established diagnosis of MS, as defined by the 2010 revision of McDonald Diagnostic Criteria [Polman C H, et al. Diagnostic criteria for multiple sclerosis: 2010 revisions to the McDonald criteria. Ann Neurol. 2011; 69(2):292-302], with relapsing course from onset (i.e., relapsing-remitting multiple sclerosis and secondary progressive multiple sclerosis [SPMS] with superimposed relapses). The trial included up to a maximum 15% of subjects with SPMS with superimposed relapses.

Subjects had active disease evidenced by one or more MS attacks with onset within the period of 12 to 1 months prior to baseline EDSS assessment, or by two or more MS attacks with onset within the 24 to 1 months prior to baseline EDSS assessment, or with one or more gadolinium-enhancing (Gd+) lesion(s) of the brain on an MRI performed within 6 months prior to baseline EDSS assessment. Enrolled subjects were ambulatory with an EDSS score of up to 5.5 inclusive. The subjects were treatment-naïve (i.e., no MS disease-modifying therapy received at any time in the past) or previously treated with interferon (IFN) β-1a, IFN β-1b, glatiramer acetate, dimethyl fumarate, or natalizumab.

### Exclusion Criteria:

Subjects with significant medical conditions or therapies for such conditions (e.g., cardiovascular, pulmonary, immunological, hepatic, ophthalmological, ocular) or lactating or pregnant women were not eligible to enter the study.

Subjects with contraindications to MRI or with clinically relevant medical or surgical conditions that, in the opinion of the investigator, would put the subject at risk by participating in the study were not eligible to enter the study.

### Study/Treatment Duration:

For an individual subject, the maximum duration of the study was approximately 118 weeks consisting of 6 weeks of screening, 108 weeks of treatment and 4 weeks of safety follow-up. Subjects discontinuing treatment prematurely had an option to stay in a post-treatment observation period (PTOP) for up to 108 weeks.

The study consisted of the following periods:

Pre-randomization period—Up to 45 days before randomization.

Treatment period: The double-blind treatment period lasted for 108 weeks. It consisted of a randomization visit, visits at two, four, and 12 weeks after randomization, and 12-weekly visits thereafter.

### End-of-Treatment (EOT):

The EOT visit took place at Week 108 (or earlier in case of premature discontinuation of study drug). In all cases, the EOT visit took place one day after the last dose of study drug but no later than 7 days after the last dose of study drug.

Subjects who completed treatment until Week 108 were eligible to enroll in an extension study conducted under a separate protocol. Subjects who discontinued study drug prematurely for any reason were not eligible for the extension study.

Subjects who prematurely discontinued study drug treatment were subsequently treated according to local standard of care at the investigator's discretion and were followed in the post-treatment observation period.

### Post-Treatment Safety Follow-Up (FU) Period:

Teriflunomide is eliminated slowly from plasma. An accelerated elimination procedure was used by all subjects after the last dose of study drug. A safety FU after the last dose of study drug was mandated.

All subjects entered the safety FU period:

For subjects who entered the extension study, the FU period started after the last dose of study drug and ended with a safety FU visit (FU1) 14-22 days after the last dose of study drug or with an abbreviated FU2 23-37 days after the last dose of study drug (if compliance to the teriflunomide accelerated elimination procedure was assessed as not sufficient at FU1).

For subjects who did not enter the extension study, the safety FU period lasted for 30 days after the last dose of study drug and included two safety FU visits (FU1, FU2) at 14-22 and 30-37 days after the last dose of study drug, respectively.

Post-treatment observation period (PTOP):

Subjects who prematurely discontinued study treatment enter the PTOP which lasts until 108 weeks after randomization (i.e., planned EOT period). It consisted of an abbreviated schedule of assessments at the time of the originally scheduled 12-weekly visits.

### End-of-Study (EOS)

EOS was reached when treatment, safety FU, and, if applicable, PTOP have been completed.

For subjects who completed the 108-week treatment period and entered the extension study, the EOS visit corresponded to the FU visit (FU1) conducted 14-22 days after the last study drug dose or to the abbreviated FU2 visit conducted 23-37 days after the last study drug dose (if needed for compliance reasons with the teriflunomide accelerated elimination procedure).

For all other subjects, the EOS visit corresponded to the 30-day FU visit (FU2) or to the last visit of PTOP (i.e., Week 108 Visit of the PTOP), whichever was last.

### Study Treatment:

The treatment period consisted of an up-titration period (from Day 1 to 14) and a maintenance period (Day 15 until EOT).

During an initial phase of the study, the study drugs in the up-titration period were administered in a double-dummy fashion. Ponesimod (or matching placebo) was presented as tablet, and teriflunomide 14 mg (or matching placebo) was presented as capsule (i.e., daily administration of one tablet and one capsule). At a later phase, the double-dummy material (tablet and capsule) was replaced by the daily administration of one capsule containing either ponesimod or teriflunomide.

In the maintenance period, the study treatment consisted of the daily administration of one capsule containing ponesimod 20 mg or teriflunomide 14 mg.

To reduce the first-dose effect of ponesimod, an up-titration scheme was implemented from Day 1 to Day 14:
Days 1 and 2; 2 mg.
Days 3 and 4; 3 mg.
Days 5 and 6; 4 mg.
Day 7; 5 mg.
Day 8; 6 mg.
Day 9; 7 mg.
Day 10; 8 mg.
Day 11; 9 mg.
Days 12, 13, and 14; 10 mg.
Day 15 until EOT; 20 mg.

Main analysis set for efficacy: The Full Analysis Set (FAS) included all randomized subjects. Subjects were evaluated according to the treatment they were randomized to.

Efficacy variable/timepoint: The endpoint was increase in ventricular volume up to the end of study (EOS). All available data up to EOS, regardless of treatment discontinuation was included (ITT approach).

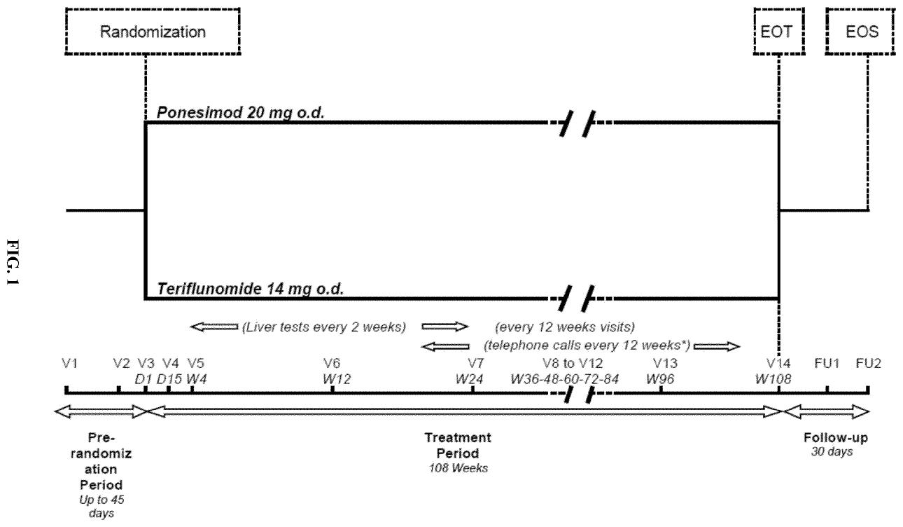

See FIG. 1 for a schematic representation of the study design.

### Statistical Methods

The Full Analysis Set (FAS) included all randomized subjects. In order to adhere to the intention-to-treat principle as much as possible, subjects were evaluated according to the treatment they have been randomized to.

The Per-Protocol Set (PPS) comprises all subjects included in the FAS without any major protocol deviations, that impact the assessment of the endpoint, occurring prior to or at randomization.

The Safety Set (SAF) included all randomized subjects who received at least one dose of study treatment. Subjects were analyzed based on actual treatment taken, not randomized treatment.

### Disposition and Baseline Characteristics:

A total of 1133 subjects were randomized to the study, 567 to ponesimod 20 mg and 566 to teriflunomide 14 mg. Overall treatment and study discontinuation were balanced across both treatment arms, 83% of subjects completed treatment. The mean age was 36.7 years and 64.9% of subjects were female. Most subjects were recruited in Europe with 50.6% from EU countries. Mean baseline EDSS score was 2.6 and mean disease duration was 7.6 years. Mean pre-study 12-month relapse rate was 1.3, and 42.6% subjects had ≥1 gadolinium-enhancing (Gd+) T1 lesions. The treatment arms were generally balanced in terms of demographics and baseline disease characteristics.

### 1. Subject and Treatment Information

A total of 1468 subjects were screened. Of those, 1133 subjects were randomized (567 to ponesimod 20 mg and 566 to teriflunomide 14 mg) across 162 sites in 28 countries, and 1131 subjects received at least one dose of study drug. The disposition of subjects is summarized in Table 1 and a summary of reasons (primary reason) for treatment discontinuation are shown in Table 2. Overall treatment and study discontinuation were balanced across both treatment arms. A total of 6.5% and 2.5% of the subjects discontinued due to AEs or tolerability related reasons in ponesimod 20 mg and teriflunomide 14 mg, respectively, while 1.9% and 4.3% discontinued due to efficacy related reasons. There were 2 deaths reported during the study—both on teriflunomide 14 mg.

### 1.1 Disposition and Treatment Discontinuation Information

TABLE 1
Disposition of subjects [Analysis Set - Subjects screened]

| | Ponesimod | | Teriflunomide | | | |
|---|---|---|---|---|---|---|
| | 20 mg | | 14 mg | | Total | |
| | N = 567 | | N = 566 | | N = 1133 | |
| | n (%) | | n (%) | | n (%) | |
| Subjects screened | | | | | 1468 | |
| Subjects re-screened | | | | | 110 | |
| Subjects randomized | 567 | (100) | 566 | (100) | 1133 | (100) |
| Subjects randomized after re-screening | 47 | (8.3) | 36 | (6.4) | 83 | (7.3) |
| Subjects treated | 565 | (99.6) | 566 | (100) | 1131 | (99.8) |
| Subjects completed treatment as per protocol | 471 | (83.1) | 473 | (83.6) | 944 | (83.3) |
| Subjects completed study as per protocol | 490 | (86.4) | 495 | (87.5) | 985 | (86.9) |
| Subjects completed treatment and study as per | 465 | (82.0) | 465 | (82.2) | 930 | (82.1) |
| protocol | | | | | | |
| Subjects stayed in study beyond safety follow-up | 67 | (11.8) | 62 | (11.0) | 129 | (11.4) |
| (PTOP) | | | | | | |

Percentages based on subjects randomized Safety follow-up is up to EOT + 30 days. PTOP = Post-treatment observation period. Output: T_DS_02_SC, Produced by birdwi1 on 2019-07-04T15:02 (CET), Data Extraction Date: 2019 Jun. 27, SDTM date: 2019 Jul. 3 Program: val_csr/program_output/T_DISP02.sas

TABLE 2
Reasons for premature treatment discontinuation [Analysis Set - Safety Set]

| | Ponesimod | | Teriflunomide | | | |
|---|---|---|---|---|---|---|
| | 20 mg | | 14 mg | | Total | |
| | N = 565 | | N = 566 | | N = 1131 | |
| | n (%) | | n (%) | | n (%) | |
| Subjects who prematurely | 94 | (16.6) | 93 | (16.4) | 187 | (16.5) |
| discontinued study treatment | | | | | | |
| Reasons for premature | | | | | | |
| discontinuation of study treatment | | | | | | |
| Subject decision | 39 | (6.9) | 49 | (8.7) | 88 | (7.8) |
| Efficacy related | 7 | (1.2) | 14 | (2.5) | 21 | (1.9) |
| Tolerability related | 8 | (1.4) | 5 | (0.9) | 13 | (1.1) |
| Other | 19 | (3.4) | 26 | (4.6) | 45 | (4.0) |
| Not known | 5 | (0.9) | 4 | (0.7) | 9 | (0.8) |
| Physician decision | 40 | (7.1) | 23 | (4.1) | 63 | (5.6) |
| Adverse event | 29 | (5.1) | 9 | (1.6) | 38 | (3.4) |
| Lack of efficacy/treatment failure | 4 | (0.7) | 10 | (1.8) | 14 | (1.2) |
| Other | 7 | (1.2) | 4 | (0.7) | 11 | (1.0) |
| Pre-specified study treatment | 12 | (2.1) | 16 | (2.8) | 28 | (2.5) |
| discontinuation criteria | | | | | | |
| Lost to follow-up | 2 | (0.4) | 3 | (0.5) | 5 | (0.4) |
| Death | 0 | | 2 | (0.4) | 2 | (0.2) |
| Reason not provided | 1 | (0.2) | 0 | | 1 | (0.1) |

Output: T_DS_05_S, Produced by birdwi1 on 2019-07-04T15:02 (CET), Data Extraction Date: 2019 Jun. 27, SDTM date: 2019 Jul. 3 Program: val_csr/program_output/DS05.sas

### 1.2 Demographic and Baseline Characteristics

Randomization was stratified by prior-DMT in the last two years prior to randomization (yes: 39.5%; no: 60.5%) and EDSS score at baseline (≤3.5: 83.3%; >3.5 16.7%). The mean age was 36.7 years and the majority of subjects (64.9%) were female. Most subjects were recruited in Europe with 50.6% from EU countries. Mean baseline EDSS score was 2.6, mean disease duration was 7.6 years and 97.4% were RRMS subjects. Mean pre-study 12-month relapse rate was 1.3, and 42.6% subjects had ≥1 Gd+T1 lesions on brain MRI. The treatment arms were generally balanced in terms of demographics and baseline disease characteristics (Tables 3 and 4).

TABLE 3
Demographic characteristics [Analysis Set - Full Analysis Set]

| | Ponesimod | | Teriflunomide | | | |
|---|---|---|---|---|---|---|
| | 20 mg | | 14 mg | | Total | |
| | N = 567 | | N = 566 | | N = 1133 | |
| Sex [n (%)] | | | | | | |
| n | 567 | | 566 | | 1133 | |
| Male | 204 | (36.0) | 194 | (34.3) | 398 | (35.1) |
| Female | 363 | (64.0) | 372 | (65.7) | 735 | (64.9) |
| Age (years) | | | | | | |
| n | 567 | | 566 | | 1133 | |
| Mean | 36.7 | | 36.8 | | 36.7 | |
| SD | 8.74 | | 8.74 | | 8.74 | |
| Median | 36.0 | | 37.0 | | 37.0 | |
| Q1, Q3 | 30.0, | 44.0 | 30.0, | 44.0 | 30.0, | 44.0 |
| Min, Max | 18, | 55 | 18, | 55 | 18, | 55 |
| Race [n (%)] | | | | | | |
| n | 567 | | 566 | | 1133 | |
| White | 551 | (97.2) | 553 | (97.7) | 1104 | (97.4) |
| American Indian or Alaska Native | 0 | | 1 | (0.2) | 1 | (0.1) |
| Black or African American | 3 | (0.5) | 2 | (0.4) | 5 | (0.4) |
| Other | 5 | (0.9) | 2 | (0.4) | 7 | (0.6) |
| Not applicable | 8 | (1.4) | 8 | (1.4) | 16 | (1.4) |
| Geographical region/Country of | | | | | | |
| enrolling site [n (%)] | | | | | | |
| European Union (EU) + UK | 289 | (51.0) | 284 | (50.2) | 573 | (50.6) |
| Europe Non-EU + Russia | 233 | (41.1) | 239 | (42.2) | 472 | (41.7) |
| North America | 32 | (5.6) | 24 | (4.2) | 56 | (4.9) |
| Rest of World | 13 | (2.3) | 19 | (3.4) | 32 | (2.8) |

Output: T_DM_01_F (Modified from original), Produced by birdwi1 on 2019-07-04T15:02 (CET), Data Extraction Date: 2019 Jun. 27, SDTM date: 2019 Jul. 3 Program: val_csr/program_output/DM01.sas

TABLE 4
Baseline disease characteristics [Analysis Set - Full Analysis Set]

| | Ponesimod | | Teriflunomide | | | |
|---|---|---|---|---|---|---|
| | 20 mg | | 14 mg | | Total | |
| | N = 567 | | N = 566 | | N = 1133 | |
| Baseline EDSS | | | | | | |
| N | 567 | | 566 | | 1133 | |
| Mean | 2.57 | | 2.56 | | 2.56 | |
| SD | 1.174 | | 1.229 | | 1.201 | |
| Median | 2.50 | | 2.50 | | 2.50 | |
| Q1, Q3 | 1.50, | 3.50 | 1.50, | 3.50 | 1.50, | 3.50 |
| Min, Max | 0.0, | 5.5 | 0.0, | 5.5 | 0.0, | 5.5 |
| Any DMT(a) received within | | | | | | |
| 2 years prior to | | | | | | |
| Randomization (eCRF) [n (%)] | | | | | | |
| N | 567 | | 566 | | 1133 | |
| Yes | 213 | (37.6) | 211 | (37.3) | 424 | (37.4) |
| No | 354 | (62.4) | 355 | (62.7) | 709 | (62.6) |
| Time since first symptoms | | | | | | |
| (years) at randomization | | | | | | |
| N | 567 | | 566 | | 1133 | |
| Mean | 7.63 | | 7.65 | | 7.64 | |
| SD | 6.781 | | 6.782 | | 6.779 | |
| Median | 5.84 | | 5.70 | | 5.77 | |
| Q1, Q3 | 2.40, | 10.97 | 2.24, | 11.03 | 2.32, | 11.01 |
| Min, Max | 0.2, | 40.8 | 0.2, | 30.8 | 0.2, | 40.8 |
| Number of relapses in last | | | | | | |
| year prior to study entry | | | | | | |
| N | 567 | | 565 | | 1132 | |
| Mean | 1.2 | | 1.3 | | 1.3 | |
| SD | 0.61 | | 0.65 | | 0.63 | |
| Median | 1.0 | | 1.0 | | 1.0 | |
| Q1, Q3 | 1.0, | 1.0 | 1.0, | 2.0 | 1.0, | 1.0 |
| Min, Max | 0, | 4 | 0, | 5 | 0, | 5 |
| Multiple sclerosis subtype [n (%)] | | | | | | |
| N | 567 | | 566 | | 1133 | |
| RRMS | 552 | (97.4) | 552 | (97.5) | 1104 | (97.4) |
| SPMS | 15 | (2.6) | 14 | (2.5) | 29 | (2.6) |
| Presence of Gd+ T1 lesions at baseline | | | | | | |
| (from central reader) [n (%)] | | | | | | |
| N | 567 | | 564 | | 1131 | |
| Yes | 226 | (39.9) | 256 | (45.4) | 482 | (42.6) |
| No | 341 | (60.1) | 308 | (54.6) | 64 | (57.4) |
| Volume of T2 lesions at baseline | | | | | | |
| [mm3] (from central reader) | | | | | | |
| N | 565 | | 563 | | 1128 | |
| Mean | 8301.4 | | 9489.2 | | 8894.3 | |
| SD | 10346.28 | | 11265.42 | | 10826.32 | |
| Median | 4841.3 | | 5651.0 | | 5171.7 | |
| Q1, Q3 | 1679.6, | 11004.4 | 2022.9, | 12978.7 | 1851.3, | 11754.1 |
| Min, Max | 0, | 86053 | 0, | 82776 | 0, | 86053 |
| Highly active disease [n (%)] | | | | | | |
| N | 567 | | 566 | | 1133 | |
| Yes | 202 | (35.6) | 200 | (35.3) | 402 | (35.5) |
| No | 365 | (64.4) | 366 | (64.7) | 731 | (64.5) |

(a) DMT = MS disease-modifying treatment.
RRMS = Relapsing-remitting multiple sclerosis, SPMS = Secondary progressive multiple sclerosis. Output: T_SC_01_F (Modified from original), Produced by birdwi1 on 2019-07-04T15:02 (CET), Data Extraction Date: 2019 Jun. 27, SDTM date: 2019 Jul. 3 Program: val_csr/program_output/SC01.sas

### 1.3 Extent of Exposure

The mean treatment exposure (irrespective of interruptions) was 96.7 weeks in the ponesimod 20 mg arm and 97.5 weeks in the teriflunomide 14 mg arm. The cumulative exposure to ponesimod 20 mg was 1045 subject-years and was 1057 subject-years for teriflunomide 14 mg arm.

TABLE 5
Study treatment exposure [Analysis Set - Safety Set]

| | Ponesimod | | Teriflunomide | |
|---|---|---|---|---|
| | 20 mg | | 14 mg | |
| | N = 565 | | N = 566 | |
| Treatment exposure, | | | | |
| irrespective of | | | | |
| interruptions (weeks) | | | | |
| N | 564 | | 566 | |
| Mean | 96.69 | | 97.45 | |
| SD | 29.018 | | 27.022 | |
| Median | 108.00 | | 108.00 | |
| Q1, Q3 | 107.29, | 108.71 | 107.29, | 108.57 |
| Min, Max | 0.3, | 111.3 | 0.1, | 113.0 |
| Treatment exposure, | | | | |
| irrespective of interruptions | | | | |
| N | 564 | | 566 | |
| Cumulative exposure (years) | 1045.2 | | 1057.1 | |

Treatment exposure based on study drug log. Treatment duration only presented for subjects with available complete treatment end date. Interruptions derived based on study drug log and number of capsules taken.
Output: T_EX_01_S(Modified from original), Produced by birdwil on 2019-07-04T15:02 (CET), Data Extraction Date: 2019 Jun. 27, SDTM date: 2019 Jul. 03 Program: val_csr/program_output/EX01.sas

### 2. Safety

### 2.1 Summary of all Adverse Events

An overview of treatment emergent AEs (TEAEs) is presented in Table 6.

TABLE 6
Overview of treatment-emergent adverse
events (AE) [Analysis Set - Safety Set]

| | Ponesimod | Teriflunomide |
|---|---|---|
| | 20 mg | 14 mg |
| | N = 565 | N = 566 |
| Characteristic | n (%) | n (%) |
| Subject with at least one | | |
| AE | 502(88.8) | 499(88.2) |
| Severe AE | 39(6.9) | 26(4.6) |
| Drug-Related AE | 278(49.2) | 238(42.0) |
| AE leading to study drug discontinuation | 49(8.7) | 34(6.0) |
| Serious AE | 49(8.7) | 46(8.1) |
| Fatal AE | 0 | 2(0.4) |

OUTPUT: T_AE_01_S, Produced by JCD on 04JUL2019 17:38 (CET), Data Extraction Date: 27 Jun. 2019, SDTM date: 3 Jul. 2019; Program: T_AE_01_S.sas

Overall, the proportion of subjects who experienced at least one TEAE was similar in both treatment arms (88.8% and 88.2% of subjects in the ponesimod 20 mg and the teriflunomide 14 mg arms, respectively).

The most common TEAEs in the ponesimod 20 mg arm were ALT increased (19.5%), nasopharyngitis (19.3%), headache (11.5%) and upper respiratory tract infection (10.6%). The most common TEAEs in the ponesimod 20 mg arm were ALT increased (19.5% vs 9.4% in the teriflunomide arm), nasopharyngitis (19.3% vs 16.8%), headache (11.5% vs 12.7%) and upper respiratory tract infections (10.6% vs 10.4%).

TEAEs leading to premature treatment discontinuation were reported in 8.7% of ponesimod 20 mg subjects compared to 6.0% of teriflunomide 14 mg subjects [see Table 7]. While the number of events was low, the difference in the type of AEs leading to treatment discontinuation was mainly driven by anticipated class effects on respiratory system and macular edema. No infections led to permanent study treatment discontinuation in the study.

TABLE 7
Treatment-emergent AEs leading to premature discontinuation
of study drug by SOC [Analysis Set - Safety Set]

| | Ponesimod | Teriflunomide |
|---|---|---|
| | 20 mg | 14 mg |
| | N = 565 | N = 566 |
| System Organ Class | n (%) | n (%) |
| Subjects with at least one AE | 49(8.7) | 34(6.0) |
| Investigations | 12(2.1) | 10(1.8) |
| Respiratory, thoracic and mediastinal | 7(1.2) | 0 |
| disorders | | |
| Eye disorders | 5(0.9) | 0 |
| Gastrointestinal disorders | 4(0.7) | 4(0.7) |
| Blood and lymphatic system disorders | 3(0.5) | 2(0.4) |
| General disorders and administration | 3(0.5) | 2(0.4) |
| site conditions | | |
| Hepatobiliary disorders | 3(0.5) | 2(0.4) |
| Pregnancy, puerperium and perinatal | 3(0.5) | 3(0.5) |
| conditions | | |
| Vascular disorders | 3(0.5) | 0 |
| Nervous system disorders | 2(0.4) | 4(0.7) |
| Social circumstances | 2(0.4) | 1(0.2) |
| Cardiac disorders | 1(0.2) | 2(0.4) |
| Musculoskeletal and connective tissue | 1(0.2) | 1(0.2) |
| disorders | | |
| Neoplasms benign, malignant and | 1(0.2) | 1(0.2) |
| unspecified (incl cysts and polyps) | | |
| Psychiatric disorders | 1(0.2) | 1(0.2) |
| Skin and subcutaneous tissue disorders | 1(0.2) | 2(0.4) |
| Reproductive system and breast disorders | 0 | 1(0.2) |
| Surgical and medical procedures | 0 | 1(0.2) |

System Organ Classes are based on MedDRA version 21.0. SOCs are sorted by descending order of frequency in the ponesimod arm.
Modified from output T_AE_18_S, Produced by AGB on 04JUL2019 17:38 (CET), Data Extraction Date: 27 Jun. 2019, SDTM date: 3 Jul. 2019, Program: T_AE_03_S_to_T_AE_23_1R.sas

There were two deaths reported in the study, one due to coronary artery insufficiency and one due to multiple sclerosis. Both deaths occurred in subjects receiving teriflunomide 14 mg.

The proportion of subjects who experienced at least one SAE was similar in both treatment arms (8.7% and 8.1% of subjects in the ponesimod 20 mg and the teriflunomide 14 mg arms, respectively).

An overview of AEs of special interest (AESIs) addressing anticipated risks of ponesimod is presented in Table 8. The most common AESIs were reported for category hepatobiliary disorders/liver enzyme abnormality (25.7% vs 14.5% in ponesimod 20 mg compared to teriflunomide 14 mg, respectively), followed by category hypertension (10.1% vs 9.0%), and pulmonary events (8.0% vs 2.7%).

TABLE 8
Treatment-emergent AESIs by category [Analysis Set - Safety Set]

| | Ponesimod | Teriflunomide |
|---|---|---|
| | 20 mg | 14 mg |
| | N = 565 | N = 566 |
| AESI Category | n (%) | n (%) |
| Hepatobiliary disorders/Liver | 145(25.7) | 82(14.5) |
| enzyme abnormality | | |
| Hypertension | 57(10.1) | 51(9.0) |
| Pulmonary events | 45(8.0) | 15(2.7) |
| Effect on heart rate and rhythm | 29(5.1) | 24(4.2) |
| (including hypotension) | | |
| Herpetic infection | 27(4.8) | 27(4.8) |
| Infection | 9(1.6) | 5(0.9) |
| Seizure | 8(1.4) | 1(0.2) |
| Macular edema | 6(1.1) | 1(0.2) |
| Skin malignancy | 5(0.9) | 1(0.2) |
| Non-skin malignancy | 1(0.2) | 1(0.2) |

Categories are sorted by descending order of frequency in the ponesimod 20 mg arm. AESI - Adverse Event of Special Interest. Infection AESI are identified by the AEs from the Infections and Infestations SOC, only if reported as serious or severe.
Modified from outputs T_AE_31_S, T_AE_38_S, T_AE_39_S, T_AE_41_S, T_AE_42_S, T_AE_43_S, T_AE_44_S, T_AE_45_S, T_AE_46_S, T_AE_48_S. All produced by JCD on 04JUL2019 17:38.

The proportion of subjects who experienced ALT increase >3×ULN was higher in the ponesimod arm (17.3%) compared to teriflunomide (8.3%) whereas ALT increase >8×ULN was higher in the teriflunomide arm (2.1%) compared to ponesimod (0.7%). Based on the individual case review, most ALT/AST increases ≥3×ULN occurred as a single transient asymptomatic episode, resolving with continued treatment or after protocol mandated treatment discontinuation. All but one case of bilirubin increase >2×ULN occurred in subjects with pre-treatment bilirubin increases. One case of potential Hy's law occurred in a subject with pre-existing transaminase elevation (ALT >5×ULN), and the event fully resolved within 2 weeks after treatment discontinuation.

The incidence of treatment-emergent heart rate and rhythm (including hypotension) AESIs on Day 1 was higher in the ponesimod 20 mg arm (2.1%) than in the teriflunomide 14 mg arm (0.4%). See Table 8A. However, the overall incidence of first dose AESI on Day 1 was low (2.1%) in ponesimod. None of these events were serious nor led to permanent discontinuation of study treatment. Discharge criteria at 4 hours post-dose were met for ca. 99% of subjects. No 2nd or higher degree AV block was observed. ECG HR effect: nadir at 2 hours post-dose (siponimod—3-4 hours, fingolimod—around by 6 hours). Low incidence of low HR outliers (post-dose HR≤40 bpm), all 3 of them with a pre-treatment HR of <55 bpm, which is a known risk factor for post-dose bradycardia with SIP receptor modulators.

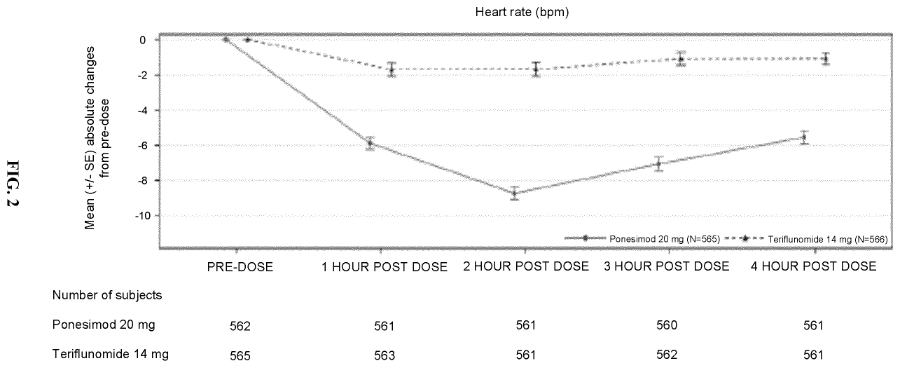

The mean heart rate reduction compared to pre-dose reached a maximum for ponesimod 20 mg at 2-hours post dose, −8.7 bpm compared to −1.7 bpm for teriflunomide 14 mg (FIG. 2). There were 3 subjects with asymptomatic post-dose HR≤40 bpm in the ponesimod 20 mg arm (none on teriflunomide 14 mg); all of these subjects had a pre-treatment HR<55 bpm, which would require post-dose monitoring according to regulatory precedence of siponimod [Mayzent® USPI].

TABLE 8A
Treatment-emergent AESI by PT: Effect on heart rate and rhythm
(including hypotension) on Day 1 [Analysis Set - Safety Set]

| | Ponesimod | Teriflunomide |
|---|---|---|
| | 20 mg | 14 mg |
| | N = 565 | N = 566 |
| Preferred Term | n (%) | n (%) |
| Subjects with at least one AE | 12(2.1) | 2(0.4) |
| Bradycardia | 4(0.7) | 0 |
| Atrioventricular block first degree | 3(0.5) | 0 |
| Defect conduction intraventricular | 2(0.4) | 0 |
| Bundle branch block left | 1(0.2) | 0 |
| Bundle branch block right | 1(0.2) | 0 |
| Sinus arrhythmia | 1(0.2) | 0 |
| Sinus bradycardia | 1(0.2) | 0 |
| Electrocardiogram QT prolonged | 0 | 1(0.2) |
| Presyncope | 0 | 1(0.2) |

Preferred Terms are based on MedDRA version 21.0.
Preferred terms are sorted by descending order of frequency in the ponesimod arm.
AESI - Adverse Event of Special Interest
OUTPUT: T_AE_32_S, Produced by JCD on 04JUL2019 17:38 (CET), Data Extraction Date: 27 Jun. 2019, SDTM date: 3 Jul. 2019, Program: T_AE_32_S_and_T_AE_33_1R.sas

### Ventricular Volume

### Objective

One objective associated with the study was to determine whether ponesimod is more efficacious than teriflunomide in terms of effects on change in ventricular volume.

Ventricular Volume (VV) data were obtained from MRI images from patients that participated in the OPTIMUM study. Data were obtained from those patients who had MRI imagines at baseline week 60 and week 108. One pre-requisite was to select only those patients who had all images completed. For patients who only had an MRI image at week 60 but not at week 108, VV was not analyzed. Hence, data were obtained from a sub-set of patients that completed the OPTIMUM study.

### Methods

Ventricular volume was estimated from MRI images collected at baseline (BL), week 60 and week 108 for 428 subjects treated with ponesimod and 427 with teriflunomide. Lesions were identified using T2-weighted and Proton Density (PD) MRIs. Ventricles were segmented using lesion-filled T1 images using with the longitudinal stream in FreeSurfer 7. For each subject, scaled ventricular volume (V) was computed as the sum of lateral and 3rd ventricle volumes divided by the intracranial volume (ICV) and expressed as a percentage. Average V (V) and average change in V (ΔV) from baseline at weeks 60 and 108 were compared across treatment arms. Ventricular volumes were correlated with Multiple Sclerosis Functional Composite (MSFC), Paced Auditory Serial Addition Test (PASAT), Average Timed 25 Foot Walk (T25FW), Average 9-Hole Peg Test (9-HPT), Single Digit Modality Test (SDMT), FSIQ-RMS and Expanded Disability Score (EDSS).

A brief description of the tests is given below. In this case, the correlation is a negative correlation, and at a group level the ventricular volume increases as the performance on these tests worsens or diminishes, e.g., indicating worse disease state.

Multiple Sclerosis Functional Composite (MSFC)—the MSFC is a three-part, standardized, quantitative, assessment instrument for use in clinical studies to measure leg function/ambulation, arm/hand function, and cognitive function.

Paced Auditory Serial Addition Test (PASAT)—the PASAT is a measure of cognitive function that assesses auditory information processing speed and flexibility, as well as calculation ability.

Average Timed 25 Foot Walk (T25FW)—the T25-FW is a quantitative mobility and leg function performance test based on a timed 25-walk.

Average 9-Hole Peg Test (9-HPT)—the 9-HPT is a standardized, quantitative test of upper extremity function.

Single Digit Modality Test (SDMT)—the SDMT detects cognitive impairment, the presence of brain damage and changes in cognitive functioning over time and in response to treatment.

Fatigue Symptoms and Impact Questionnaire—Relapsing Multiple Sclerosis (FSIQ-RMS)—the FSIQ-RMS is an MS specific 20-item PRO measure that comprises 2 domains: one measuring MS symptoms (7 items) and one measuring MS-related impacts (13 items).

Expanded Disability Score (EDSS)—the EDSS is an ordinal clinical rating scale ranging from 0 (normal neurologic examination) to 10 (death due to MS) in half-point increments.

### Results

At baseline, week 60 and week 108, scaled ventricular volume (V) for all subjects correlated with MSFC, PASAT, T25FW, 9-HPT, SDMT and EDSS (all p-values <0.0001) and FSIQ-RMS (p value <0.05). The results are summarized in FIGS. 3-6.

FIG. 3 demonstrates that ventricular enlargement is less at week 60, baseline, and week 108, baseline, in subjects treated with ponesimod (n=428) compared to teriflunomide (n=427).

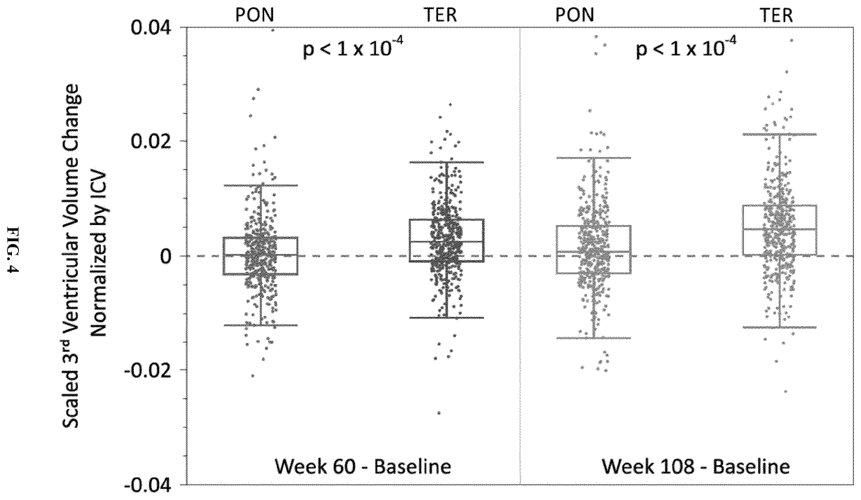

FIGS. 4-6 demonstrate that reduction of atrophy by ponesimod (n=428) is greater compared to teriflunomide (n=427), as illustrated by the difference in longitudinal increase in the size of the 3rd ventricle and the lateral ventricle at week 60 and week 108 compared to baseline.

The significant correlations indicate that larger ventricular volumes are associated with worse clinical outcomes. V was similar (p=0.861) between treatment arms at 1.595% (SD 0.91), and 1.581% (SD 0.77) for ponesimod and teriflunomide, respectively. In both arms, average ventricular volumes expressed as a percentage of ICV, increased from baseline throughout the double blind period: Δ₆₀V^{pon}=0.026%, p<0.001, Δ₆₀V^{ter}=0.065%, p<0.001, Δ₁₀₈V^{pon}=0.067%, p<0.001, Δ₁₀₈V^{ter}=0.124%, p<0.001.

These increases however were significantly lower in the ponesimod arm, compared to the teriflunomide arm at both week 60 (Δ₆₀ V^{pon}−Δ₆₀V^{ter}=−0.025%, p<0.001) and week 108 (Δ₁₀₈V^{pon}−Δ₁₀₈V^{ter}=0.043%, p<0.001).

### CONCLUSION

Increase in ventricular volume throughout the double-blind phase was observed for both ponesimod and teriflunomide treated patients. However, the rate of increase on ponesimod was 60% lower at week 60 and 45% lower at week 108 compared to teriflunomide, suggesting a reduced rate of neurodegeneration on ponesimod, as measured by MRI-based ventricular volume.

1. A method of slowing an increase in brain ventricular volume in a patient in need thereof, comprising administering ponesimod to the patient using a regimen that is effective to slow the increase in brain ventricular volume.

2. The method of claim 1, wherein the brain ventricular volume is determined by magnetic resonance imaging.

3. The method of claim 1, wherein the patient has multiple sclerosis (MS).

4. The method of claim 1, wherein the multiple sclerosis is relapsing multiple sclerosis.

5. The method of claim 4, wherein the relapsing multiple sclerosis comprises relapsing-remitting disease, clinically isolated syndrome or active secondary progressive disease.

6. The method of claim 1, wherein about 20 mg of ponesimod is administered orally once daily.

7. The method of claim 1, wherein the regimen comprises an up-titration step at initiation of the method or upon re-initiation of the method after a discontinuation, comprising administering orally once daily 2 mg of ponesimod on days 1 and 2: 3 mg of ponesimod on days 3 and 4: 4 mg of ponesimod on days 5 and 6: 5 mg of ponesimod on day 7: 6 mg of ponesimod on day 8: 7 mg of ponesimod on day 9; 8 mg of ponesimod on day 10; and 9 mg of ponesimod on day 11: 10 mg of ponesimod on days 12, 13, and 14, followed by the administering of the 20 mg of ponesimod once daily thereafter.

8. The method of claim 1, wherein the patient has a baseline expanded disability status scale (EDSS) score of less than or equal to 3.5.

9. The method of claim 1, wherein the patient has a baseline expanded disability status scale (EDSS) score of less than or equal to 3.

10. The method of claim 1, wherein slowing the increase in brain ventricular volume is relative to a patient population having substantially similar baseline disease characteristics and receiving a standard of care treatment that does not comprise ponesimod.

11. The method of claim 10, wherein the standard of care treatment comprises teriflunomide.

12. The method of claim 10, wherein the standard of care treatment comprises administration of about 14 mg of teriflunomide orally once daily.

13. The method of claim 1, wherein the relative slowing of the increase in ventricular volume is at least about 40%.

14. The method of claim 1, wherein the relative slowing of the increase in ventricular volume is at least about 60%.

15. The method of claim 14, wherein the relative slowing of the increase in ventricular volume results within about 60 weeks from initiation of treatment with ponesimod.

16-45. (canceled)
